(19) United States
(12) Patent Application Publication
(10) Pub. No.: US 2006/0168699 A1
(43) Pub. Date: Jul. 27, 2006

(54) Method for preparing transformed cucumis melo

(76) Inventors: Sun Lee, Daejon City (KR); Je-Geun Yoo, Daejon City (KR)

Correspondence Address: NIXON & VANDERHYE, PC, 901 NORTH GLEBE ROAD, 11TH FLOOR, ARLINGTON, VA, 22203, US

(21) Appl. No.: 10/501,087

(22) PCT Filed: Aug. 1, 2002
(86) PCT No.: PCT/KR02/01462

§ 371 Date: Apr. 11, 2005

(30) Foreign Application Priority Data
Jan. 9, 2002 (KR) 2002/1149

(51) Int. Cl. C12N 15/82; A01H 5/00

(52) U.S. Cl. 800/294; 800/308

(57) ABSTRACT

The present invention relates to a method of preparing a transformed Cucumis melo using Acrobacterium tumefaciens, more particularly, to a method for preparing a transformed Cucumis melo, which comprises the steps of: (a) inoculating a cotyledon from Cucumis melo with Agrobacterium tumefaciens harboring a suitable vector; (b) regenerating the inoculated cotyledon in a regeneration medium containing 3.0-8.0 mg/l of kinetin as growth regulator and 0.5-3.0 mg/l of IAA (Indole-3-acetic acid) and culturing the inoculated cotyledon to obtain regenerated shoots; and (c) culturing the regenerated shoots on a rooting medium to obtain the transformed Cucumis melo.

### BACKGROUND OF THE INVENTION

### Field of the Invention

The present invention relates to a plant transformation, more particularly, relates to a method for preparing a transformed Cucumis melo L using Acrobacterium tumefaciens and a transformed Cucumis melo L prepared therefrom.

Together with a watermelon, Cucumis melo L. is a representative summer fruit in Korea, of which the area under cultivation is about 10,859 ha and the yield is about 320 thousand ton (Ministry of Agriculture and Forestry in Korea, 1999). The domestic seed market of it amounts to 8,548 liter, that corresponds to about 6 billion wons (Korean Seed Association, 1999).

Recently, many crops that are high value-added using transformation technologies come into the market (Ann, M. T. Transforming agriculture. Chemical & Engineering April:21-35(1999)). Although the transformation of other crops of Cucurbitaleae such as watermelons, melons and cucumbers has been reported (Choi, P. S. et al., Genetic transformation and plant regeneration of watermelon using Agrobacterium tumefaciens. Plant Cell Rep. 13:344-348(1994); Dong J. Z. et al., Transformation of melon(Cucumis Melo L.) and expression from the cauliflower mosaic virus 35S promoter in transgenic melon plants. Biotechnology 9:858-863(1991); and Lee K. W. et al., Expression of TMV Coat Protein Gene in Transgenic Tobacco and Cucumber Plants Using Binary Vector System of Agrobacterium tumefaciens. Korean Journal of Plant Tissue Culture 23(4)205-210(1996)), the transformation of Cucumis melo has not been developed yet.

Therefore, referring to the redifferentiation and transformation of Cucumis melo, many efforts are required to be made. The development of a transformation technology of Cucumis melo is the most fundamental that can accommodate needs of consumers promptly and can produce high-quality melons(Cucumis melo) with lower cost.

Throughout this application, various publications are referenced and citations are provided in parentheses. The disclosure of these publications in their entities are hereby incorporated by references into this application in order to more fully describe this invention and the state of the art to which this invention pertains.

### SUMMARY OF THE INVENTION

Under such situation, the present inventors have made intensive research to resolve the need in the art and as a result, we have completed the present invention by establishing a novel method for transformation of Cucumis melo such as a germination condition of seeds, a coculturing method with Agrobacterium tumefaciens and a unique composition of a regeneration medium. According to the present method, a preparation of a transformed Cucumis melo with Agrobacterium tumefaciens could be done more effectively in shorter time.

Accordingly, it is an object of this invention to provide a method for preparing a transformed Cucumis melo using Agrobacterium tumefaciens.

It is another object of this invention to provide a transformed Cucumis melo prepared with the Agrobacterium tumefaciens.

Other objects and advantages of the present invention will become apparent from the detailed description to follow taken in conjunction with the appended claims.

### BRIEF DESCRIPTION OF THE DRAWINGS

FIG. 1 represents a genetic map of binary vector pRD320 used in this invention;

FIG. 2 represents a photograph showing the results of GUS analysis confirming the occurrence of transformation in Cucumis melo;

FIG. 3 represents a gel photograph showing the results of PCR elucidating transformed Cucumis melo according to this invention; and

FIG. 4 represents a gel photograph showing the results of Southern blotting confirming the occurrence of transformation in Cucumis melo.

### DETAILED DESCRIPTION OF THE INVENTION

In one aspect of this invention, there is provided a method for preparing a transformed Cucumis melo, which comprises the steps of: (a) inoculating a cotyledon from Cucumis melo with Agrobacterium tumefaciens harboring a vector, in which the vector is capable of inserting into a genome of a cell from Cucumis melo and contains the following sequences: (i) a replication origin operable in the cell from Cucumis melo; (ii) a promoter capable of promoting a transcription in the cell from Cucumis melo; (iii) a structural gene operably linked to the promoter; and (iv) a polyadenylation signal sequence; (b) regenerating the inoculated cotyledon in a regeneration medium containing 3.0-8.0 mg/l of kinetin as growth regulator and 0.5-3.0 mg/l of IAA (Indole-3-acetic acid) and culturing the inoculated cotyledon to obtain regenerated shoots; and (c) culturing the regenerated shoots on a rooting medium to obtain the transformed Cucumis melo.

In order to obtain an effective method of transformation for Cucumis melo, the present inventors have made extensive research with 4 cultivars of Cucumis melo developed in Korea, and finally developed methods for regeneration with higher probability and transformation with Agrobacterium tumefaciens in more efficient manner, as exemplified in Examples below.

The present invention will be described in more detail as follows:

I. Preparation of Starting Material for Transformation

The preferred explant for transformation includes leaf, stem and petiole, but not limited to. The explant may be obtained from several plant organs and most preferably from seed. It is preferred that the seed is sterilized with sterilizing agent such as chlorine and chlorides (e.g., sodium hypochloride) before use.

II. Seed Germination

According to a preferred embodiment of this invention, the medium for seed germination comprises nutrient basal medium such as MS, B5, LS, N6 and White's, energy source and vitamins, but not limited to. Sugars are useful as energy source and sucrose is the most preferable. It is preferred that vitamins for seed germination include nicotine, thiamine and pyridoxine. In addition, the medium for seed germination in this invention may further contain MES (2-(N-Morpholino) ethanesulfonic acid Monohydrate) as buffering agent for pH change and agar as solid support. The medium is unlikely to contain plant growth regulators.

III. Preparation of Plant Tissue for Transformation

In this invention, the explant for transformation includes any tissue derived from seed germinated. It is preferred to use cotyledon and hypocotyl and the most preferred is cotyledon. It is advantageous to remove growth point completely from cotyledon as explant.

IV. Inoculation with Agrobacterium tumefaciens

Transformation of cells derived from Cucumis melo is carried out with Agrobacterium tumefaciens harboring Ti plasmid (Depicker, A. et al., Plant cell transformation by Agrobacterium plasmids. In Genetic Engineering of Plants, Plenum Press, New York (1983)). More preferably, binary vector system such as pBin19, pRD400 and pRD320 is used for transformation (An, G. et al., Binary vectors” In Plant Gene Res. Manual, Martinus Nijhoff Publisher, New York(1986)).

The binary vector useful in this invention carries: (i) a replication origin operable in the cell from Cucumis melo; (ii) a promoter capable of promoting a transcription in the cell from Cucumis melo; (iii) a structural gene operably linked to the promoter; and (iv) a polyadenylation signal sequence. In addition to this, it is preferred that the vector carries antibiotics-resistance gene as selective marker, e.g. carbenicllin, kanamycin, spectinomycin and hygromcin. The vector may alternatively further carry a gene coding for reporter molecule (for example, luciferase and β-glucuronidase). Examples of the promoter used in the binary vector include but not limited to Cauliflower Mosaic Virus 35S promoter, 1′ promoter, 2′ promoter and promoter nopaline synthetase (nos) promoter. The structural gene in the present vector may be determined depending on traits of interest. Exemplified structural gene may include but not limited to genes for herbicide resistance (e.g. glyphosate, sulfonylurea), viral resistance, vermin resistance (e.g., Bt gene), resistance to environmental extremes (e.g. draught, high or low temperature, high salt conc.), improvement in qualities (e.g. increasing sugar content, retardation of ripening), exogenous protein production useful as drug (EGF, antigen or antibody to various diseases, insulin) or cosmetic raw material (e.g. albumin, antibiotic peptide).

Inoculation of the explant with Agrobacterium tumefaciens involves procedures known in the art. The preferred inoculation is carried out in such a manner that after dissecting the cotyledon with growth point removed, the cotyledon sections are immersed to coculture in culture of Agrobacterium tumefaciens, thereby inoculating the cotyledon with Agrobacterium tumefaciens. Preferably, 3.0-8.0 mg/l of kinetin, 0.5-3.0 mg/l of IAA (Indole-3-acetic acid) and 50-200 μM of acetosyringone are employed in the inoculating medium. Kinetin and IAA is employed to regulate the growth and acetosyringone to promote infection of Agrobacterium tumefaciens into explant cell.

V. Regeneration

It is necessary that explant tissue, which is transformed with Agrobacterium tumefaciens, be regenerated in a regeneration medium with strictly controlled ingredients and quantities thereof. The regeneration medium of this invention may contain nutrient basal medium such as MS, B5, LS, N6 and White's, energy source and vitamins, but not limited to. Sugars are useful as energy source and sucrose is the most preferable. It is preferred that vitamins in regeneration medium include nicotine, thiamine and pyridoxine. In addition, the regeneration medium may further contain MES (2-(N-Morpholino) ethanesulfonic acid Monohydrate) as buffering agent for pH change and agar as solid support.

The medium must contain plant growth regulators. Cytokinin as plant growth regulator may include but not limited to 6-benzylaminopurine (BAP), kinetin, zeatin and isopentyladenosine and kinetin is the most preferable cytokinin. Furthermore, the regeneration medium contains, as essential ingredient, auxin such as NAA (α-naphthalene acetic acid), indole acetic acid (IAA), (2,4-dichlorophenoxy) acetic acid, and the most preferable is IAA.

Preferably, the amount of kinetin in the regeneration medium ranges from 5.0 to 7.0 mg/l, and the amount of IAA ranges from 1.0 to 2.0 mg/l.

The regeneration medium of this invention further may comprise CuSO4 or casein hydrolysate known to promote regeneration in addition to the growth regulators, and preferable is CuSO₄. The most suitable amount of CuSO₄ ranges from 0.5 to 2.0 mg/l.

According to a preferred embodiment of this invention, the medium further contains antibiotics (e.g. carbenicllin, kanamycin, spectinomycin or hygromcin) for selection of transformed explant.

Most preferably, the culture in regeneration medium is carried out under the following conditions: 25±1° C.; 16 hr:8 hr (light culture : dark culture); and 4,000 lux of illumination intensity.

Culturing in accordance with the conditions described above allows successfully a regeneration of shoots through callus formation from the transformed explant of Cucumis melo on the medium.

VI. Rooting

The transformed Cucumis melo plantlet is finally produced on rooting medium by rooting of regenerated shoots. The rooting medium of this invention may contain nutrient basal medium such as MS, B5, LS, N6 and White's, energy source and vitamins, but not limited to. Sugars are useful as energy source and sucrose is the most preferable. It is preferred that vitamins in the rooting medium include nicotine, thiamine and pyridoxine. In addition, the rooting medium may further contain MES (2-(N-Morpholino) ethanesulfonic acid Monohydrate) as buffering agent for pH change and agar as solid support.

As plant growth regulator, auxin is predominantly employed in the rooting medium. The auxin useful includes α-naphthalene acetic acid (NAA), indole acetic acid and (2,4-dichlorophenoxy) acetic acid, and the most preferable is NAA. Preferably, the amount of NAA in the rooting medium is in the range of from 0.08 to 0.2 mg/l.

VII. Confirmation of Transformation

The transformed Cucumis melo produced according to the present invention may be confirmed using procedures known in the art. For example, using DNA sample from tissue of transformed Cucumis melo, PCR is carried out to reveal exogenous gene incorporated into a genome of Cucumis melo transformed. Alternatively, Northern or Southern Blotting may be performed for confirming the transformation as described in Maniatis et al., Molecular Cloning, A Laboratory Manual, Cold Spring Harbor Laboratory, Cold Spring Harbor, N.Y. (1989).

According to the most preferred embodiment of this invention, there is provided a method for preparing a transformed Cucumis melo, which comprises the steps of: (a) inoculating an cotyledon from Cucumis melo in an inoculating medium containing 3.0-8.0 mg/l of kinetin, 0.5-3.0 mg/l of IAA and 50-200 μM of acetosyringone with Agrobacterium tumefaciens harboring a vector, in which the vector is capable of inserting into a genome of a cell from Cucumis melo and contains the following sequences: (i) a replication origin operable in the cell from Cucumis melo; (ii) a promoter capable of promoting a transcription in the cell from Cucumis melo; (iii) a structural gene operably linked to the promoter; and (iv) a polyadenylation signal sequence, (b) regenerating the inoculated cotyledon in a regeneration medium containing 5.0-7.0 mg/l of kinetin and 1.0-2.0 mg/l of IAA as growth regulators and CuSO4 as a regenerating accelerating agent to obtain regenerated shoots; and (c) culturing the regenerated shoots to obtain the transformed Cucumis melo on a rooting medium containing 0.08-0.2 mg/l of NAA.

In another aspect of the present invention, there is provided a transformed Cucumis melo prepared by the methods of this invention described above.

The method of this invention, which is developed for producing a transformed Cucumis melo, as exemplified and demonstrated in Examples below, exhibits much higher transformation and regeneration efficiency, leading to production of transformed Cucumis melo having desirable traits with higher reproducibility.

The following specific examples are intended to be illustrative of the invention and should not be construed as limiting the scope of the invention as defined by appended claims.

### EXAMPLE 1

### Preparing of Explants

4 cultivars of Cucumis melo (SagyejoelKul, KeumnodajiEuncheon, HwangjineeEuncheon and KeumhwangEuncheon), which have been developed in Korea, were employed in regeneration and transformation experiments. Seed coats from seeds of 4 cultivars kept at 4° C. were removed with physical method and sterilized for 30 min in 1% NaOCl solution and washed 4 times with sterilized DW. The sterilized seeds were dried and seeded on seeding media (containing ½ MSMS, 1.0% sucrose and 0.6% agar and pH 5.6) then cultured to germinate seed for 1 week at 26±1° C., 4,000 lux and 16 hrs/8 hrs (day/night) under light condition. Thereafter, the resulting cotyledons were used as samples.

### EXAMPLE 2

### Transformation of Explant Tissue

The growth point from cotyledons in Example 1 was completely removed.

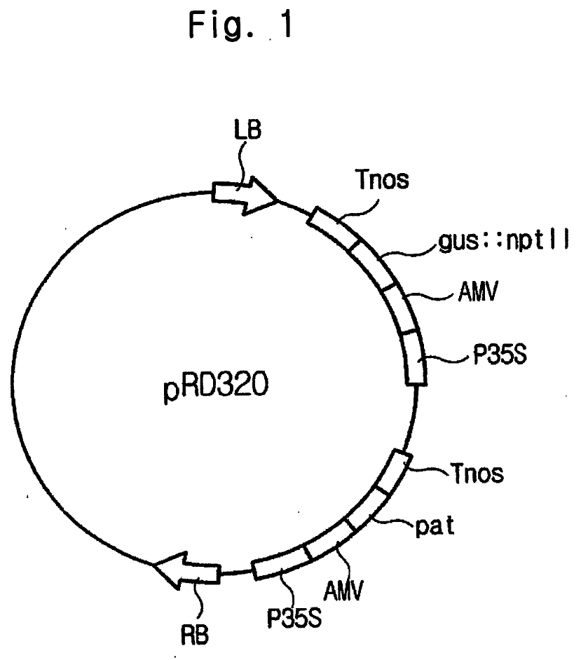

Agrobacterium tumefaciens (Agrobacterium tumefaciens GV3101(Mp90) ; Plant-cell-rep., 15(11):799-803(1996)) transformed with binary vector pRD320 shown in FIG. 1 (Omirulleh,-S. et al., Activity of a chimeric promoter with the doubled CaMV 35S enhancer element in protoplast-derived cells and transgenic plants in maize. Plant. Mol. Biol. Int. J. Mol. Biol. Biochem. Genet. Eng., 21(3):415-428(1993)) was cultured in super broth containing 100 μM of acetosyringone (37 g/l brain heart infusion broth(Difco) and 0.2% sucrose, pH 5.6) at 28° C. for 18 hours, then the resulting medium was diluted 20-fold with inoculation medium. The inoculation medium (pH 5.6) contains MSB5 (Murashige & Skoog medium including Gamborg B5 vitamins), 3.0% sucrose, 0.5 g/l of MES [2-(N-Morpholino)ethanesulfonic acid Monohydrate], 6.0 mg/l of kinetin, 1.5 mg/l of IAA, 1.0 mg/l of CuSO₄·5H₂O, 100 μM of acetosyringone and 5% DMSO (dimethylsulfoxide). In FIG. 1, LB and RB represent left and right border of T-DNA, respectively and MCS, Tnos, P35S, AMV, nptII, gus::nptII and pat represent multiple cloning site, termination sequence of nos, CaMV (Cauliflower mosaic virus) 35S enhancer, Alfalfa mosaic virus enhancer, glucuronidase::neomycin phosphotransferase fusion gene, and gene encoding phosphinotrysin acetyltransferase, respectively.

Thereafter, the cotyledon was immersed in 40 ml of an inoculation medium and cultured for 20 minutes to inoculate with Agrobacterium tumefaciens. Then, the cotyledon was transferred to a coculturing medium with its outface being upward. The coculturing medium contains MSB5, 3.0% sucrose, 0.5 g/l of MES, 6.0 mg/l of kinetin, 1.5 mg/l of IAA, 1.0 mg/l of CuSO₄.5H₂O, 0.6% agar, 100 μM of acetosyringone and 5% DMSO. Thereafter, the cotyledon was cocultured under dark culture condition (26±1° C., 24 hrs night) for 3 days.

### EXAMPLE 3

### Regeneration

After coculturing, to form shoots by regeneration from cotyledon and to select transformed shoots, the cotyledon was placed on a regeneration medium (selection medium) and cultured at 25±1° C. and 4,000 lux under 16 hour light condition to induce generation of shoots. The regeneration medium (pH 5.6) contains MSB5, 3.0% sucrose, 0.5 g/l of MES, 6.0 mg/l of kinetin, 1.5 mg/l of IAA, 1.0 mg/l of CuSO₄.5H₂O, 0.6% agar, 100 mg/l of kanamycin and 500 mg/l of carbenicillin. Then, the regenerated shoots were transferred to a fresh selection medium followed by light culture for 2 weeks.

Thereafter, the elongated shoots were transferred to a rooting medium and cultured for 2 weeks. The shoots with roots, which were considered to be transformed, were selected. The rooting medium (pH 5.6) contains MSB5, 3.0% Sucrose, 0.5 g/l of MES, 0.1 mg/l of NAA (α-naphtalene acetic acid), 1.0 mg/l of CuSO₄.5H₂O, 0.6% agar, 100 mg/l of kanamycin and 500 mg/l of carbenicillin.

The transformation rates are indicated in Table 1.

TABLE 1

| | No. | Gus (+) | Transform |
|---|---|---|---|
| Cultivar | of Explant | Shoot¹⁾ | (%)²⁾ |
| A³⁾ | 290 | 12 | 4.1 |
| B⁴⁾ | 310 | 43 | 13.9 |
| C⁵⁾ | 300 | 19 | 6.3 |
| D⁶⁾ | 360 | 0 | 0 |

¹⁾number of shoots developing blue color in GUS analysis of Example 4,
²⁾transformation rate calculated by [(GUS (+) shoot number/explant number) × 100],
³⁾KeumhwangEuncheon,
⁴⁾KeumnodajiEuncheon,
⁵⁾HwangjineeEuncheon, and
⁶⁾SagyejeolKul

As shown in Table 1, the cultivar exhibiting the highest transformation rate (14.7%) is KeumnodajiEuncheon. Such high transformation rate has not been reported for Cucumis melo in any publication. In addition, the average transformation rate of KeumhwangEuncheon, KeumnodajiEuncheon and HwangjineeEuncheon is revealed to be relatively high (8.2%).

### EXAMPLE 4

### Confirmation of Transformant

The transformants produced in Example were confirmed as follows:

### EXAMPLE 4-1

### GUS Analysis

To measure activity of β-glucuronidase, the samples to be tested were immersed in X-GluA (5-Bromo-4-Chloro-3-Indole-β-D-Glucuronic Acid) solution (100 mM NaPO₄, pH 7.0, 3 mM K₃[Fe(CN)6], 10 mM EDTA, 0.1% Triton X-100, and 2 mM X-GluA) and treated in vacuum for 10 minutes and reacted at 37° C. for one night. The reacted tissues were placed in 0.5% NaCl solution to remove chlorophyll and observed the staining level and shape with naked eye.

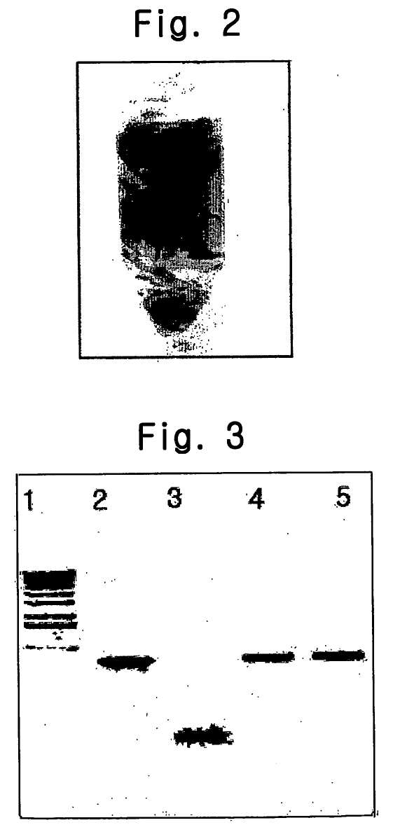

Cucumis melo was likely to generate non-transformed shoots in selection medium much more compared with other crops such as Nicotiana tabacum or Arabidopsis thaliana. However, there is no elongation of roots. The results of GUS activity of highly elongated shoots showed strong gene expression in leaves, tendrils and stems (FIG. 2).

### EXAMPLE 4-2

### PCR Analysis

10 mg of shoots selected for transformation in the selection medium were crushed in 100 μl of 0.5 M NaOH solution and 500 μl of 100 mM Tris (pH 8.0) solution were added to. 1.0 μl of DNA obtained from Tris solution with the crushed shoots was used as PCR template. PCR primers were nptII primers (sense primer: 5′-GAT GGA GTG CAC GCA GGT-3′ and antisense primer: 5′-TCA GAA GAA CTC GTC AAG-3′). 50 ng of template DNA, 1.0 μl of 10× PCR reaction buffer (100 mM Tris.HCl, pH 8.8, 1 5 mM MgCl₂, 50 mM KCl and 1% Triton X-100), 2.0 μl of dNTPs (each 2.5 mM), 0.25 μl (100 pmol) of the sense and the antisense primer and 0.25 μl (2 Unit) of Taq DNA polymerase were prepared and sterilized DW was added to 25 μl of the final volume.

The PCR was performed in such a manner that pre-denaturation at 94° C. for 2 minutes was done and total 35 cycles were done in which each cycle is composed of denaturation at 94° C. for 30 seconds, annealing at 55° C. for 30 seconds and extension at 72° C. for 2 minutes, followed by final extension at 72° C. for 10 min and kept at 4° C. The amplified products were loaded on 1.0% agarose gel for electrophoresis with TBE electrophoresis solution at 50-100 V for 60-90 minutes. The gel was immersed in 0.002% EtBr for 20 minutes, washed with DW and transferred to UV illuminator to determine molecular weight (FIG. 3). In FIG. 3, lanes 1, 2, 3 and 4 represent size marker, and PCR products of genomic DNAs from KeumhwangEuncheon, from SagyejoelKul, from KeumnodajiEuncheon and from HwangjineeEuncheon, respectively.

It was demonstrated that NptII gene in the regenerated shoots of KeumhwangEuncheon, KeumnodajiEuncheon and HwangjineeEuncheon was amplified with PCR to obtain 800 bp of nptII DNA fragment, and there was no amplified gene in non-transformed SagyejoelKul showing negative GUS reaction.

### EXAMPLE 4-3

### Southern Blot Analysis

Referring to manuals for ECL kit provided by Amersham, Southern blotting was performed. Two grams of the plant tissues were crushed with liquid nitrogen in mortar and 5 ml of lysis buffer were added. Then, 40 μl of 1% proteinase K and 2.5 ml of 10% SDS were added and incubated for 1 hr at 60° C. 2.5 ml of 5 M NaCl and 2 ml of 10% CTAB were added and stirred slowly, incubated in incubator for 1 hr at 60° C., and centrifuged at 8000×g and 5° C. for 5 min. The supernatant was transferred to other tube, the same volume of methylene chloride:isoamylalchol (24:1) were added and mixed, followed by centrifugation at 8000×g and 5° C. for 10 min. The supernatant was transferred to other tube and 20 μl of RNase A (10 mg/l) were added and incubated for 30 min at 37° C. The same volume of methylene chloride:isoamylalchol (24:1) were added and centrifuged at 8000×g and 5° C. for 10 min. The supernatant was transferred to other tube and the same volume of ice-stored isopropanol was added and mixed. Following centrifugation at 8000×g and 5° C. for 15 min., the supernatant was discarded and DNA pellet was rinsed with 70% ethanol.

After drying overnight at room temperature, sterilized DW was added to dissolve DNA and DNA was digested with HindIII for 24 hr at 37° C., followed by electrophoresis on 0.8% agarose gel (0.5× TBE). The gel was incubated in 0.25 M HCl solution until the color of bromophenol blue became yellow and the solution was discarded, followed by rinsing with DW. The denaturation solution (NaCl 1.5 M and NaOH 0.5 M) was added to the gel and incubated for 30 min with slow agitation. The solution was discarded and the gel was rinsed with DW. Then, the neutralizing solution (NaCl 1.5 M, Tris/HCl 0.5 M, pH 7.5) was added and incubated with agitation for 30 min.

Upon the completion of incubation, the solution was discarded and rinsed with DW, and the neutralizing solution was added, followed by additional incubation with agitation for 30 min. Thereafter, 3MM paper, gel, nylon membrane, 3MM paper sheet, paper towel and weight were placed in order over the glass plate filled with 20×SSC, and capillary blotting was performed. Pat gene tagged with ³²P-dCTP was employed as probe in hybridization.

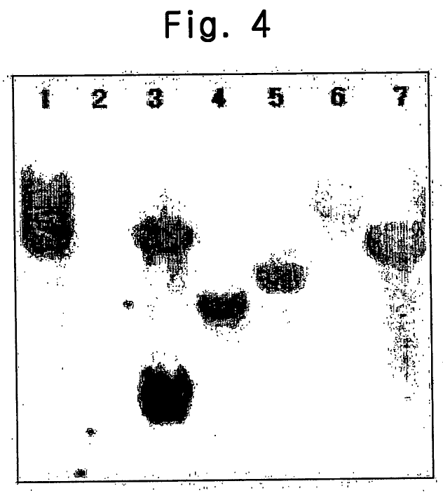

As shown in FIG. 4, the southern blotting revealed that among 3 explants from KeumhwangEuncheon being subject to transformation, each of 2 explants had two exogenous genes (lanes 1 and 3) and 1 explant was not transformed (lane 2). In addition, it was observed that 2 explants from KeumnodajiEuncheon (lanes 4 and 5) and HwangjineeEuncheon (lanes 6 and 7) had one exogenous gene.

In conclusion, according to the present invention, novel Cucumis melo with desirable traits can be obtained with higher regeneration and transformation rate.

1. method for preparing a transformed Cucumis melo, which comprises the steps of:
(a) inoculating a cotyledon from Cucumis melo with Agrobacterium tumefaciens harboring a vector, in which the vector is capable of inserting into a genome of a cell from Cucumis melo and contains the following sequences:
(i) a replication origin operable in the cell from Cucumis melo; (ii) a promoter capable of promoting a transcription in the cell from Cucumis melo; (iii) a structural gene operably linked to the promoter; and (iv) a polyadenylation signal sequence,
(b) regenerating the inoculated cotyledon in a regeneration medium containing 3.0-8.0 mg/1 of kinetin as growth regulator and 0.5-3.0 mg/1 of IAA (Indole-3-acetic acid) and culturing the inoculated cotyledon to obtain regenerated shoots; and
(c) culturing the regenerated shoots on a rooting medium to obtain the transformed Cucumis melo.

2. The method according to claim 1, wherein an amount of kinetin in the regeneration medium of step (b) is 5.0-7.0 mg/l.

3. The method according to claim 1, wherein an amount of IAA in the regeneration medium of step (b) is 1.0-2.0 mg/l.

4. The method according to claim 1, wherein an amount of NAA in the rooting medium of step (c) is 0.08-0.2 mg/l.

5. The method according to claim 1, wherein the step (a) is executed in an inoculating medium containing 3.0-8.0 mg/l of kinetin, 0.5-3.0 mg/1 of IAA and 50-200 μM of acetosyringone.

6. The method according to claim 1, wherein the regeneration medium further comprises 0.5-2.0 mg/l of CuSO₄.

7. A method for preparing a transformed Cucumis melo, which comprises the steps of:
(a) inoculating an cotyledon from Cucumis melo with Agrobacterium tumefaciens harboring a vector in an inoculating medium containing 3.0-8.0 mg/l of kinetin, 0.5-3.0 mg/l of IAA and 50-200 μM of acetosyringone, in which the vector is capable of inserting into a genome of a cell from Cucumis melo and contains the following sequences:
(i) a replication origin operable in the cell from Cucumis melo; (ii) a promoter capable of promoting a transcription in the cell from Cucumis melo; (iii) a structural gene operably linked to the promoter; and (iv) a polyadenylation signal sequence,
(b) regenerating the inoculated cotyledon in a regeneration medium containing 5.0-7.0 mg/l of kinetin and 1.0-2.0 mg/l of IAA as growth regulators and CuSO4 as a regenerating accelerating agent to obtain regenerated shoots; and
(c) culturing the regenerated shoots to obtain the transformed Cucumis melo on a rooting medium containing 0.08-0.2 mg/l of NAA.

8. A transformed Cucumis melo prepared by the method according to claim 1.
